(19) United States
(12) Patent Application Publication
(10) Pub. No.: US 2010/0113984 A1
(43) Pub. Date: May 6, 2010

(54) Geometrically shaped hydrogel standoffs for coupling high intensity focused ultrasound

(76) Inventors: Joseph A. Leonetti, Daly City, CA (US); Larry L. Smith, Lummi Island, WA (US)

Correspondence Address: ROBERT L. MCDOWELL, 1170 JACKSON HEIGHTS DR, WEBSTER, NY, 14580-9367, US

(21) Appl. No.: 12/589,892

(22) Filed: Oct. 30, 2009

Related U.S. Application Data
(62) Division of application No. 11/213,279, filed on Aug. 26, 2005, now abandoned.
(60) Provisional application No. 60/604,784, filed on Aug. 26, 2004.

(51) Int. Cl. A61N 7/00

(52) U.S. Cl. 601/2

(57) ABSTRACT

In vivo biocompatible hydrogels to couple and transmit high intensity ultrasound for hemostasis and ablation during surgery, the hydrogels having 4-40 wt. % of a polymer comprising acrylates and the balance water. A group of hydrogels based on cross-linked methacrylate one of which is polyethyleneglycol methacrylate, can form rigid, low acoustic attenuation coupling members and are in vivo biocompatible. These coupling members consist of hydrogel formulations having mechanical and acoustic properties such that ultrasound transmission standoff members of various dimensions and structural configurations function as efficient ultrasound transmission media and devices within which the ultrasound beam can be transferred to a focal point at the end of the standoff or in close proximity to it.

This application claims the benefit of U.S. Provisional Patent Application No. 60/604,784 filed Aug. 26, 2004.

### FIELD OF THE INVENTION

The present invention is directed to ultrasound coupling devices and in particular, to geometrically shaped coupling standoffs consisting of hydrogels for use with high intensity focused ultrasound.

### BACKGROUND OF THE INVENTION

High Intensity Focused Ultrasound (HIFU) has been reported by many as a means of destroying tissue by thermal means, whereby, the tissue is heated to a temperature that denatures the tissue proteins and by mechanical means through disruption of cellular and nuclear membranes caused by localized cavitation. Others have reported the potential for HIFU to rapidly introduce hemostasis (the coagulation of blood and termination of bleeding) during surgery.

The energy requirements for HIFU to cause the therapeutic effects of hemostasis and ablation are on the order of 1,000 to 10,000 Watts/cm². Furthermore, the ultrasound energy most useful for establishing hemostasis and ablation with HIFU is in the frequency range of 2-9 MHz, which attenuates quickly in most solid materials including metals and plastics.

It is advantageous, in designing surgical tools based on HIFU, to have the zone of peak ultrasound energy to occur at or near the surface of the surgical tool so that the use is similar to other devices used for coagulation and ablation during surgery. Devices such as the electro-cautery knives and argon beam coagulators employ thermal techniques to produce hemostasis and cause ablation at the surface of the surgical tool where it contacts the patient.

One technology for producing high intensity zones useful for hemostasis and ablation is to focus ultrasound energy by means of a lens or curved piezoelectric element. This technique of focusing HIFU requires a coupling medium, typically solid or liquid, between the piezoelectric transducer and the target tissue with sufficient length (typically 1 to 6 cm) to support the transfer of the ultrasound to develop the necessary spatial peak intensity.

An acoustic coupling member is an important component of a HIFU surgical device for reasons that include:
1. It is the medium within which acoustic energy is transferred to a point of focus at or in close proximity to the end of the geometric standoff into a small focal zone, typically in the range of 1-2 mm diameter by 6-10 mm long, and at high intensity, typically over 1,000 watts/cm².
2. It can be designed so that the focal zone, is positioned either at the surface of the distal tip of the coupling member (which contacts the tissue or blood vessel) or beyond the tip at a deeper location in the tissue.
3. It can be sterilized and provided as a disposable device that can be replaced during and between surgeries.
4. It must be in vivo biocompatible, as required by its contact with blood and tissue during surgery.

Preferably, a coupling member possesses characteristics that include:
1. Low cost to manufacture into various geometric shapes including but not limited to cones, cylinders and flat membranes.
2. Have low acoustic attenuation in the frequency range of 2-9 MHz enabling efficient coupling of the high intensity focused ultrasound generated by the transducer into the target tissue.
3. Be uniform in acoustic properties so that the acoustic wave generated by the transducer is not distorted in an unpredictable manner by the coupling member.
4. Have an acoustic impedance that is similar to that of tissue and/or blood, thereby allowing the maximum transfer of acoustic energy from the coupling member into the body
5. Be produced from materials that are compatible with tissue and blood for both short and long terms (in vivo biocompatible).
6. Be robust in nature, so as to support HIFU with no degradation.
7. Be easily and quickly replaceable during the surgical procedure.

Several materials and techniques have been reported for producing HIFU coupling members. For example:

1. Water
Water meets all the desired acoustic properties required by a coupling member including the requirement of low attenuation and in vivo biocompatibility. Water is, however, difficult to contain in a manner that permits use as a coupling member for a HIFU surgical tool; whereby, the containment method does not in itself alter or negate the desirable characteristics of the water or rupture and cause the device to fail during use with subsequent difficulty in replacing the water coupling member.

2. Metals
Solid metal, including aluminum or titanium, HIFU coupling cones are robust and have been reported to address the containment problems of water in the construction of HIFU coupling members. Their disadvantages are high manufacturing cost, and high acoustic attenuation and impedance, which results in low energy transfer and the generation of unacceptable amounts of heat in the device.

3. Hydrogels
Hydrogels offer an attractive combination of the desirable acoustic properties approaching water, as they may be comprised of greater than 60% water, and the advantage of a solid material that does not have the containment problems of water. They are typically moldable, inexpensive to produce and can be quickly changed during a surgical procedure.

Hydrogels have been used as coupling members and specifically as HIFU coupling members. However, hydrogels previously investigated as coupling members were not suitable for use during surgery due to issues of in vivo biocompatibility and/or lack of mechanical strength and resistance to HIFU degradation.

For example, polyacrylamide (PA) has been used as an acoustic coupling member for HIFU. However, polyacrylamide is not an acceptable polymer due to the potential presence of neurotoxic acrylamide monomer in the hydrogel. Acoustic coupling hydrogel standoffs produced from poly (2-hydroxyethylmethacrylate) or pHEMA have been found less suitable due to their mechanical properties and high attenuation, which is also true of hydrogels produced from alginate derivatives and polysaccharides.

### SUMMARY OF THE INVENTION

The present invention is directed to the production and use of acoustic transmission gels and semi-solid geometries from in vivo biocompatible hydrogels, in particular those derived from the acrylate family, including methacrylates and cyanoacrylates, for use with high energy focused ultrasound (HIFU).

### BRIEF DESCRIPTION OF THE DRAWINGS

FIG. 1 illustrates a preferred embodiment comprising a geometrically shaped coupling hydrogel standoff in the shape of a cone.

FIG. 2 illustrates the geometrically shaped acoustic coupling hydrogel standoff of FIG. 1 whereby the acoustic coupling member is contained within a external retention capsule which is attached to a transducer housing.

### DETAILED DESCRIPTION OF THE PREFERRED EMBODIMENT

Described below is the formulation, design and fabrication of hydrogels that possess the acoustic, mechanical and structural properties required to function as ultrasound coupling and transmission media as is used in high intensity focused ultrasound (HIFU) applications such as hemostasis and ablation during surgery. Unless noted otherwise, all percentage compositions referred to are weight percent (wt %).

The device of this invention relates to the manufacture, composition and use of in vivo biocompatible hydrogel acoustic coupling standoffs for transfer of high intensity ultrasound to achieve hemostasis and ablation during surgery. More specifically, this invention relates to the discovery that a group of hydrogels, based on alkyl methacrylates, that form rigid, coupling members possessing low acoustic attenuation and in vivo biocompatibility. These inventive devices consist of hydrogel formulations having mechanical and acoustic properties such that ultrasound coupling standoff members of various dimensions and structural geometric configurations, such as cones and flat membranes, can function as efficient ultrasound transmission media and devices within which the high intensity ultrasound beam is coupled between the acoustic energy source to a focal point at or in proximity to the standoff terminus. Hydrogel formulations, design and fabrication methods are described for production of ultrasound and energy transmission elements as the device of this invention.

The present invention is broadly directed to a family of acrylate hydrogels, including methacrylate and cyanoacrylate, and manufacturing techniques that result in geometric shaped HIFU coupling members that meet the requirements imposed for acoustic hemostasis and ablation within the human body. These requirements include:

in vivo biocompatibility

low acoustic attenuation at HIFU frequencies

ability to be easily molded into shapes

relatively low manufacturing cost

acoustic impedance similar to that of tissue and blood

relatively robust, not brittle, durable during the surgical and HIFU procedure

easy to replace during or between surgical procedures

sterilizable

Selection of hydrogels for coupling elements is based on polymer in vivo biocompatibility with subsequent evaluation of conformance to mechanical and acoustic property requirements necessary to form and function. High intensity focused ultrasound (HIFU) utilizes high frequency sound, typically between 2 and 9 MHz. Acoustic energy at such frequencies is poorly transmitted by air and requires an acoustic coupling member, typically a solid or liquid, between the transducer and the tissue. Acoustic coupling media have commonly been fluids, gels, or solids to efficiently transfer the acoustic energy between the HIFU applicator and the target tissue.

The inventive hydrogel acoustic coupling element operates as a geometric standoff between the transducer and the object of therapy. As used in HIFU applications, the high frequency acoustic energy is concentrated into a small volume (typically in the shape of a grain of rice 7-10 mm in length) and at high intensity (typically over 1,000 watts/cm²). The hydrogels thus used for such ultrasound energy transmission must provide low levels of attenuation to limit heating within the coupling element, and efficiently transfer the energy to the treatment site. The hydrogel thus used must also be thermally robust at the HIFU acoustic intensities, be in vivo biocompatible, relatively inexpensive, sterilizable and moldable into various geometries, such as cones.

By design, the cast hydrogel coupling elements, such as cone shapes, are configured so that the base of the acoustic coupling element physically and intimately conforms to the contours of the transducer face. In practice, the HIFU coupling members of this invention are secured to the transducer face so as to maintain a conformal and air free interface between the two. Such conformal interface produces an acoustic coupling between the ultrasound transducer and the hydrogel HIFU coupling member, thus providing for the transmission of the ultrasound energy at or proximate to the site of device contact with tissue, blood or blood vessels.

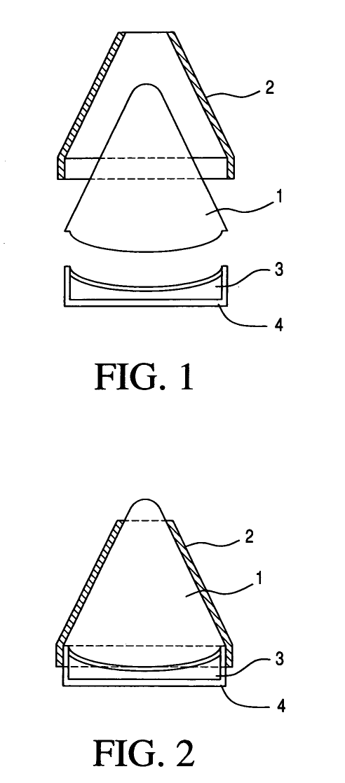

FIGS. 1 and 2 show an embodiment of the invention comprising an acoustic coupling hydrogel standoff 1 which preferably is a solid free standing hydrogel coupling member requiring no restraint or alternatively held within a retainer 2 for secure attachment and intimate contact interface to the face of a transducer 3 and its housing 4.

The mechanical and structural requirements imposed by rigid self-supporting hydrogel focus members limit the selection of suitable polymers for HIFU applications. When hydrogel acoustic coupling standoffs are designed so as to incorporate use of acoustically transparent shells or containment devices, other polymers, such as soft gels and/or semi-solids, become candidates for the standoffs. Such acoustically transparent devices can function as molds in the casting process and/or as a retainer device when in use during therapy.

In vivo biocompatible hydrogels suitable for use in HIFU application include, for example; methylmethacrylates, blends of collagen/poly (acrylic acid), collagen and poly (HEMA), PMMA and PDMS. Geometrically shaped HIFU coupling standoffs of inventive device be prepared from poly(methacrylamide), poly(hydroxyalkyl methylacrylates) such as poly(glyceryl methacrylate), poly(vinyl alcohol) crosslinked with poly(ethylene glycol) diacrylate, block copolymers composed of poly(ethylene oxide)-poly(propylene oxide)-poly(ethylene oxide) and poly(propylene oxide)-poly(ethylene oxide)-poly(propylene oxide) blocks. Such aforementioned polymers can be cross-linked with compounds such as ethylene glycol dimethacrylate or methylene-bis-acrylamide.

The most preferred family of polymers, as the device of this invention, are alkyl/alyl methacrylates that are cross-linked such as to compose in vivo biocompatible rigid hydrogel geometries that efficiently couple and transfer high intensity ultrasound between the transducer face and the treatment site. The inventive hydrogel comprises 4-40 wt. % polymer and the balance water. Preferably, the inventive hydrogel comprises 5-30% polymer and the balance water and most preferably, the inventive hydrogel comprises 8-25% polymer and the balance water.

Production of hydrogels suitable for HIFU applications focused primarily on the methacrylate compounds composed of polyethyleneglycol methacrylate, 2-hydroxyethylmethacrylate and the cross-linkers ethyleneglycol dimethacrylate, polyethyleneglycol dimethacrylate and glycerol propoxylate all of which are commercially available and purchased from Sigma-Aldrich, St. Louis, Mo. Other potential base methacrylates and cross-linkers for the device of this invention include but are not limited to acrylate, cyanoacrylate, methyl methacrylate, ethyl methacrylate, butyl methacrylate diethylaminoethyl methacrylate, and higher alkyl methacrylates, and triethyleneglycol dimethacrylate, hexanediol dimethacrylate and polyethyleneglycol dimethacrylates of various molecular weights as cross-linkers.

Ammonium persulfate was added to the solutions as an oxidizer and generator of free radicals followed by addition of N,N,N,N-tetramethylethylenediamine (TMED) to increase the rate of polymerization. Other radical initiators that can be used include AIBN (azobisisobutyronitrile) and benzoyl peroxide.

Parameters used in development process included total polymer concentration, polymer and cross-linker compositions and the ratio of base polymers to cross-linkers. Resultant samples were subjectively evaluated regarding mechanical properties including compressive strength, flexibility, fracture resistance and clarity. For initial assessment of properties, 15% polymer solutions were prepared using polyethyleneglycol methacrylate as the base polymer to which the cross linkers ethyleneglycol dimethacrylate, polyethyleneglycol dimethacrylate and glycerol propoxylate were added. Polyethyleneglycol methacrylate to cross-linker ratios in the range of 50:1 to 4:1 were evaluated for each of the cross-linkers. Subsequently, 15% polymer solutions of 2-hydroxyethylmetacrylate and cross-linkers were prepared in the same base polymer to cross-linker ratios and evaluated in the same manner and combination of cross-linkers as the polyethyleneglycol methacrylate polymer.

Castings were made in five piece molds that formed a cone shaped inner cavity. Base monomer/cross-linker solutions were first blended in water to create a 15% polymer solution to which was first added 0.84% of a 10% solution of an aqueous ammonium persulfate solution and just prior to casting a 0.06% aliquot of 99% TMED. Onset of polymerization was visualized by observed gelling of the polymer solution residing in the mold reservoir. At room temperature, polymerization to a mechanical strength sufficient to remove the castings from the molds requires approximately ten minutes from the time at which the accelerator is added to the polymer blend.

Additional sample sets were prepared with total polymer concentrations of 10, 20, 25 and 30 wt. %, with a total polymer concentration of 15% as preferred and 20% being the most preferred. Acoustic coupling members with polymer concentrations below 10% tended to become fragile as the lower end of the range (i.e. 4%) is neared. Whereas, concentrations in excess of 30% produced coupling members that tended to exceed required mechanical properties and adversely affect acoustic attenuation as the upper end of the range (i.e. 40%) is neared.

Castings prepared from 2-hydroxyethyl methacrylate cross-linked with ethyleneglycol dimethacrylate, polyethyleneglycol dimethacrylate and glycerol propoxylate did not meet the mechanical and physical property requirements for coupling standoffs due to lack of rigidity and opacity.

Evaluation of castings prepared from 20% polymer solutions indicated that the most preferred formulation for the device of this invention is polyethyleneglycol methacrylate as the base polymer and polyethyleneglycol dimethacrylate as the cross-linker. The preferred ratio of base polymer to cross linker is 15:1 and the most preferred ratio is 8:1. The most preferred initiator/accelerator system for this family is composed of ammonium persulfate and NNN′N′-tetramethylethylenediamine but is not limited thereto.

Hydrogel focus cones for HIFU applications preferred in the embodiment of this invention are produced from the methacrylate family of polymers which are cross linked in water as the base solvent.

While this invention has been described with reference to medical or therapeutic ultrasound applications with human tissue as a target, it is not to be limited thereto. The present invention is also contemplated with other animal tissue such as in veterinary ultrasound therapy. The present invention is also intended to include other suitable hydrogel polymers and modifications which would be apparent to those skilled in the art and to which the subject matter pertains without deviating from the spirit and scope of the appended claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A method of coupling and transferring high-energy ultrasound between an ultrasound transducer and a focal point at, or external to, a tip of a standoff and acoustic coupling element, said method comprising:
providing a standoff and acoustic coupling element having a defined geometric shape and comprising a hydrogel having 4-40 wt. % of a polymer comprising acrylates and the balance water,
acoustically coupling said standoff and acoustic coupling element to said ultrasound transducer,
transferring high-energy ultrasound between the ultrasound transducer and the focal point at, or external to, the tip of said standoff and acoustic coupling element.

18. (canceled)

19. (canceled)

20. (canceled)
